(19) United States
(12) Patent Application Publication
(10) Pub. No.: US 2006/0058613 A1
(43) Pub. Date: Mar. 16, 2006

(54) Respiration measurement apparatus

(76) Inventor: Hiroaki Nakai, Kanagawa-ken (JP)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/186,979

(22) Filed: Jul. 22, 2005

(30) Foreign Application Priority Data
Jul. 22, 2004 (JP) 2004-214965

(51) Int. Cl. A61B 5/05

(52) U.S. Cl. 600/407

(57) ABSTRACT

To provide a respiration measurement apparatus for measuring respiration of a human subject by using an image pickup device to ensure the privacy of the human subject. Respiration is measured by separating image pickup elements in an image pickup device connected to a respiration measurement apparatus into nonfunctional light receiving sections and functional light receiving sections, and generating an image that ensures the privacy of the human subject.

This application claims the benefit of Japanese Patent Application No. 2004-214965, filed on Jul. 22, 2004, the contents of which are incorporated in their entirety herein by reference.

### BACKGROUND

1. Technical Field

This invention relates generally to measuring respiration of a human subject by using an optical sensor.

2. Description of the Related Art

In accordance with rapid aging in recent years, as well as interest in obstructive sleep apnea syndrome and sudden infant death syndrome (SIDS), there is a need for a respiration measurement apparatus capable of automatically measuring respiration of a human subject during sleeping, which can be used to support the care of elderly persons and children, or screening of a pulmonary disease.

One respiration measurement apparatus for medical diagnosis uses a contact-type sensor, such as a mat, having a piezoelectric sensor mounted thereon, to generate a signal indicative of respiration during sleep. However, this apparatus is large, typically expensive, and requires physical contact with the human subject. Thus, this apparatus is inconvenient to a human operator and the human subject whose respiration is being measured.

Another apparatus, capable of measuring respiration without physically contacting the human subject, has been disclosed in Japanese Patent No. 3263035 B2 to Ishihara et al. (“Ishihara et al.”). A change of images due to respiration is extracted from images of the human subject during sleeping that are photographed by a camera, such as a charge-coupled device (CCD) camera, by image processing. However, since the images of the sleeping human subject are photographed by a camera, the human subject may have concerns about his or her privacy.

Yet another apparatus, which projects a predetermined lighting pattern such as a spot light to the human subject and measures his or her respiration from movement of these images, is described in Japanese Laid-Open Patent Application No. 2002-175582 to Aoki et al. The privacy of the human subject is protected upon normal usage thereof. However, this apparatus is typically expensive because a lighting device is needed to project a light pattern. Furthermore, when using the lighting device normally placed in a room, the sleeping human subject is also photographed such that the human subject's privacy is not ensured.

Thus, it is desirable to have a respiration measurement apparatus capable of measuring the respiration of the human subject while ensuring the privacy of the human subject.

### SUMMARY

The present invention has been made taking the foregoing problems into consideration and an object of which is to provide a respiration measurement apparatus capable of physically ensuring the privacy of a human subject.

A first aspect of the present invention may provide a respiration measurement apparatus, at least including: image pickup means to photograph a human subject and provide information representative of an image of the human subject; image inputting means to input the image information of the photographed human subject; change detecting means to detect a change of the image due to respiration of the human subject from the inputted image information; and respiration waveform generating means to obtain a respiration waveform from a time series of the detected change of image; wherein a plurality of non-photographable image areas is distributed in photographable image areas in the image pickup means, and the change detecting means detects the change of the image from only at least one of the photographable image areas.

In a second aspect, a respiration measurement apparatus comprises an image pickup device including an image pickup element to photograph a human subject and provide information representative of an image of the human subject. Image inputting means inputs the image information of the photographed human subject. Change detecting means detects a change of the image due to respiration of the human subject from the inputted image information. Respiration waveform generating means obtains respiration waveform from a time series of the detected change of image.

In one embodiment of the second aspect, a resolution of the image photographed by the image pickup element of the image pickup device is defined from several tens to several thousands of pixels.

In another embodiment of the second aspect, the respiration measurement apparatus includes display means to only display brightness information for individual pixels or for groups of pixels of the image pickup element in the image photographed by the image pickup device.

In yet another embodiment of the second aspect, a plurality of non-photographable image areas are distributed in photographable image areas in the image pickup element of the image pickup device, and the processor detects the change of the image only from at least one of the photographable image areas.

In a third aspect, a respiration measurement apparatus comprises an image pickup device to photograph a human subject and provide information representative of an image of the human subject. A respiration measurement device inputs the image information of the photographed human subject. A processor, included in the respiration measurement device, detects a change of the image due to respiration of the human subject from the inputted image information and generates a respiration waveform from a time series of the detected change of the image.

In one embodiment of the third aspect, a plurality of non-photographable image areas are distributed in photographable image areas in an image pickup element of the image pickup device, and the processor detects the change of the image only from at least one of the photographable image areas.

In another embodiment of the third aspect, a resolution of the image photographed by the image pickup element of the image pickup device is defined from several tens to several thousands of pixels.

In yet another embodiment of the third aspect, the respiration measurement apparatus includes display means to only display brightness information for individual pixels or for groups of pixels of the image pickup element in the image photographed by the image pickup device.

In a fourth aspect, a display device is capable of displaying brightness information for a plurality of pixels or groups of pixels of an image pickup element in an image photographed by an image pickup device of a respiration measurement apparatus. The display device comprises a plurality of indicator lights arranged in a lattice, wherein the individual indicator lights correspond to the individual pixels or groups of pixels of the image pickup element.

### BRIEF DESCRIPTION OF THE DRAWINGS

FIG. 1 is a schematic diagram of a respiration measurement apparatus using an image pickup device according to a first embodiment of the present invention.

FIG. 2 is a schematic structure of an image pickup element.

FIG. 3 illustrates a method for setting a functional light receiving section and a nonfunctional light receiving section.

FIG. 4 is an example of an image that is photographed by image pickup elements including functional pixels and nonfunctional pixels.

FIG. 5 is an illustration of an exemplary image that is photographed by image pickup elements including functional pixels and nonfunctional pixels.

FIG. 6 is an illustration of an exemplary embodiment of a light shielding mask for setting functional pixels and nonfunctional pixels.

FIG. 7 is an illustration of an exemplary embodiment of a mask setting a photographable area of an image pickup element.

FIG. 8 is an illustration of an exemplary embodiment of setting the mask for setting the photographable area outside the image pickup device.

FIG. 9 is an illustration of an exemplary embodiment of a display device for displaying an amplitude of a respiration signal.

FIG. 10 is a graph showing plots of an image change amount D(t) and a respiration waveform R(t) that are measured in accordance with respiration.

### DESCRIPTION OF THE EMBODIMENTS

Hereinafter, each embodiment of the present invention will be described with reference to the drawings.

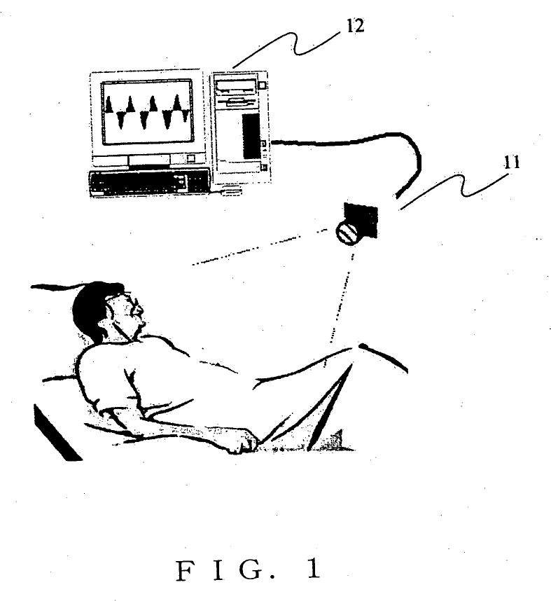

A respiration measurement apparatus according to a first embodiment will be described with reference to the exemplary embodiments illustrated in FIGS. 1 to 6.

As shown in FIG. 1, the respiration measurement apparatus according to the first embodiment is composed of at least an image pickup device 11 and a respiration measurement device 12. This structure is described below.

Specifically, the respiration measurement device 12 is an image processor that, for example, can realize the following functions by means of a computer-readable program stored on a computer-readable medium. The computer-readable medium can be read by a computer embodying the image processor to execute the computer-readable program stored thereon.

These functions include: an image input function to input the image of a human subject to be monitored, who is photographed by the image pickup device 11; a change detection function to detect change of image due to respiration from the inputted images; a respiration waveform generation function to obtain a respiration waveform from a time series of the detected change of image; and an alert issuing function to issue an alert if a cessation or abnormality of respiration is detected in the generated respiration waveform.

The above-described change detection function will be described below.

In an image area (AREA) set in a chest area of the human subject being photographed by the image pickup device 11, it is possible to detect a change in the image due to respiration by the following method. Specifically, assuming that a pixel gradation value of a position (x, y) inside the image area is defined as I_{xy}, an image

$$D ( t ) = {\sum }_{( x , y ) ∈ AREA} \vert {I}_{xy} ( t ) - {I}_{xy} ( t - K ) \vert$$

change amount D(t) at a time t is obtained as a sum of inter-frame absolute value differences in the image area according to the following equation:

If the chest area of the human subject is photographed in the image area, the image change amount D(t) may be approximately proportional to a displacement of a surface of the chest due to the respiration. An indication light can be continuously lit or a volume of an audible signal can be increased or decreased in accordance with this image change amount D(t), to inform a caretaker of a respiration state of the human subject, to record the respiration state, or to establish communication.

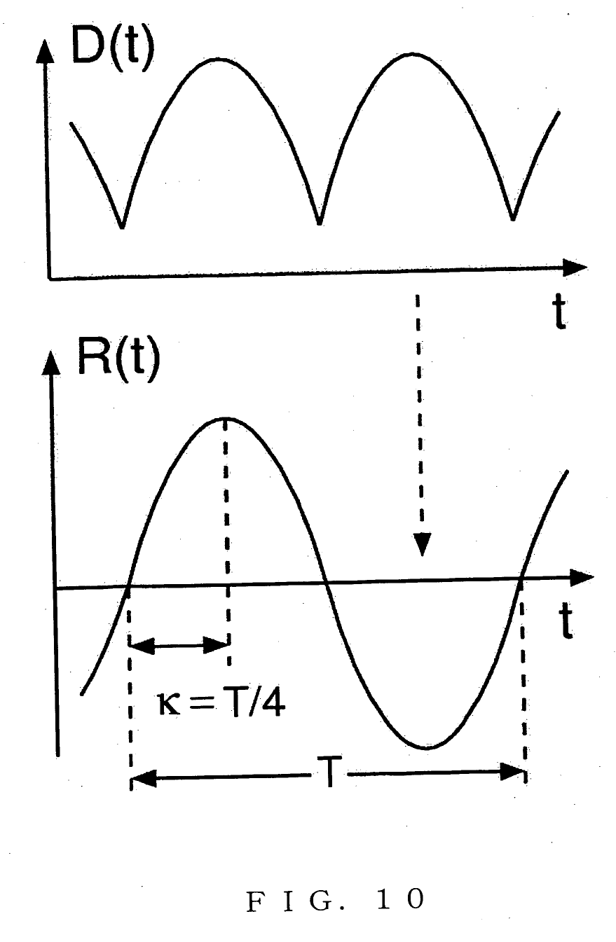

Next, the above-described respiration waveform generation function as an additional function will be described below. The image change amount D(t) is made very small when inspiration and expiration of the human subject are switched. As shown in FIG. 10, by inverting the waveform of the image change amount D(t) from positive to negative at this timing when the amount is very small, it is possible to obtain a respiration waveform R(t).

In addition, from a zero crossing interval of this substantially periodic respiration waveform R(t), a respiration cycle T can be easily measured. Here, by automatically setting a frame interval k of the inter-frame absolute value difference detected by the change detection function, for example, at ¼ of the respiration cycle T that was measured last time or the like, the change of image due to respiration is discerned from a noise background in an image pickup system, particularly in a dark environment, so that the respiration can be measured more accurately. Further, in the case that an index value indicating the condition of the respiration of the person to be cared for, such as when the measured respiration cycle T and the amplitude of the respiration waveform R(t) deviates from a normal range, by issuing an alert to inform the caretaker of this abnormality, it is possible to easily realize the alert issuing function as the additional function.

Here, the image area to be set in the chest area of the human subject may be set at a predetermined position by the caretaker or the operator or as described in Ishihara et al. and Nakai et al., “Respiration Monitoring System by Moving Image Processing,” Institute of Electronics, Information and Communication Engineers, Article Magazine D II, Vol. J83 -D-II, No. 1, pp. 280 to 288, 2000. This image area may be automatically set by automatically detecting the range having the largest change of image from among the photographed images. In other words, it is possible to arbitrarily set the image area.

Thus, it is possible to measure the respiration of the human subject without contacting him or her.

The image pickup device 11 may be a CCD camera or another apparatus capable of receiving an optical image and outputting data in relation to the optical image. Furthermore, the image pickup device 11 may be positioned to enable receiving the image of the body of the human subject to measure the respiration of the human subject.

In order to reduce psychological privacy concerns of the human subject, a countermeasure to ensure his or her privacy is taken, such as to output only the measured respiration waveforms and the alert signals from the respiration measurement device 12. However, for issue of the alert or maintenance of the device, it is inevitable that the respiration measurement device 12 is connected to the outer device through some communication device such as a network in practice, so that a possibility that the image of the human subject inputted in the respiration measurement device 12 leaks to the outside of the respiration measurement device 12 cannot be denied. Accordingly, embodiments consistent with the present invention include a mechanism to protect a right of privacy of the human subject, which is mounted on the image pickup device 11 before the image is transmitted to the respiration measurement device 12.

In the image pickup device 11, if the image of the human subject cannot be photographed in the same detail as a normal CCD camera, identifying information or behavioral information about the human subject cannot be acquired, and the structure of the apparatus and the information acquired in operation are presented to the human subject so as to satisfy him or her, the privacy concerns of the human subject can be largely satisfied. In the present specification, “non-photographable image area” means the state that the light is intentionally shielded to the pixel or the sensitivity is low so that the image cannot be photographed. A method of obtaining the image information necessary for measurement of respiration while ensuring the privacy of the human subject is described below.

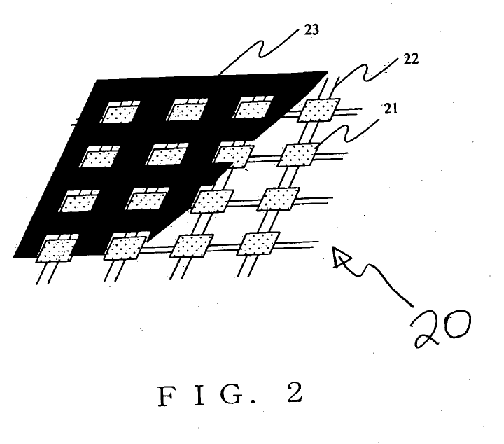

The structure of the image pickup element of the CCD camera that can be used as the image pickup device 11 will first be described generally with reference to FIG. 2.

In FIG. 2, an image pickup element 20 includes a plurality of light receiving sections 21. Each light receiving section 21 is a photodiode or any other suitable light-sensitive element (forming one pixel), and light receiving sections 21 are arranged in a lattice. Additionally, a peripheral circuit 22 for reading brightness information, or alternatively luminance information, measured by the light receiving sections 21 and a light shielding mask 23 are arranged as shown in FIG. 2 so that light can only enter the light receiving sections 21. In order to show the state of arranging the light receiving sections 21, FIG. 2 shows the state that the light shielding mask 23 is cut to form apertures in the light shielding mask 23. One light receiving section 21, the peripheral circuits connected to the light receiving section 21, and one aperture of the light shielding mask 23 corresponding to this light receiving section 21 form one pixel.

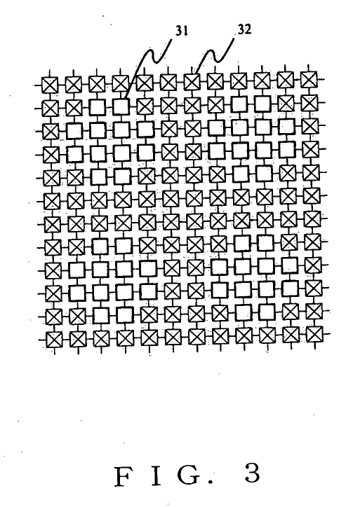

In FIG. 3, a portion of 12×12 pixels is illustrated from among many light receiving sections that are arranged in a lattice in the image pickup element 20. Here, from among the light receiving sections, functional light receiving sections 31 having a normal function (namely, pixels represented by empty squares in FIG. 3) and non-functional light receiving sections 32 processed in a manufacturing process of the image pickup element so that these non-functional light receiving sections 32 do not function normally (namely, pixels represented by X marks inside squares) are picked up. In FIG. 3, the functional light receiving sections 31 are arranged to form a plurality of groups, each group having an approximately circular shape.

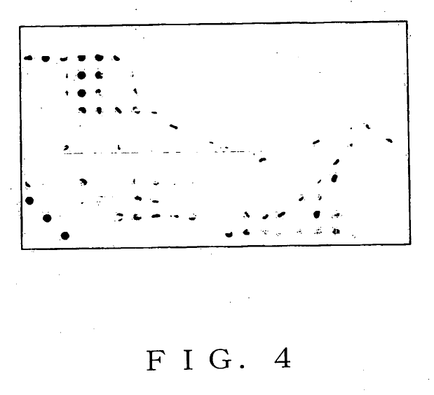

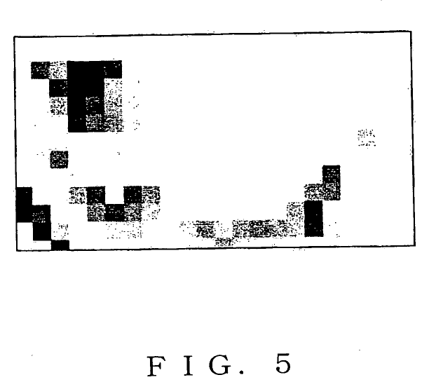

Thus, in the case that some light receiving sections are processed to be non-functional light receiving sections 32, even if the human subject is photographed as shown in FIG. 1, the image shown in FIG. 4 is obtained from this image pickup device 11, such that it is difficult to obtain the individual information such as the face and the behaviors of the human subject. Furthermore, since the location of the functional light receiving sections 31 of each group are predetermined, if the outputs of the functional light receiving sections 31 of the same group are unified, the image of the human subject obtained from this image pickup device can be made into a mosaic image as shown in FIG. 5 and this may be advantageous to ensure privacy.

Thus, even if the obtained image is limited in detail or scope of the human subject's body, it is not necessary to photograph the body of the human subject in detail and the respiration can be measured from a change in the image area of the partial body over time, so that the respiration can be measured based on the image as shown in FIG. 4 or FIG. 5. Accordingly, the respiration measurement apparatus can be realized to ensure the privacy of the human subject by providing the functional pixels and the nonfunctional pixels in the image pickup element 20.

Here, as a method of processing some pixels such that those pixels do not function as the light receiving sections 32 shown in FIG. 3, the following methods are available.

According to a first method, the light receiving section 21 is not formed in the process of manufacturing the image pickup element, such as shown in FIG. 2.

According to a second method, the light receiving section 21 is not connected to the peripheral circuit 22 in the process of manufacturing the image pickup element, such as shown in FIG. 2.

According to a third method, after forming the light receiving section 21 and the peripheral circuit 22, a function of the light receiving section 21 or the peripheral circuit 22 is disabled by electrical or mechanical operation.

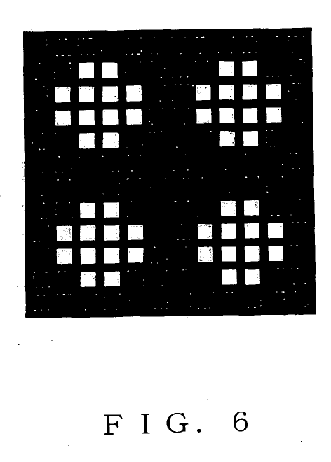

According to a fourth method, the light shielding mask 23 is used to shield some light receiving sections, such as shown in FIG. 6.

According to a fifth method, a processor for signal processing is incorporated in many image pickup devices such as a CCD camera and software for the processor for signal processing is set so as not to output a signal of specific pixels.

According to the methods described above, the functional pixels and the nonfunctional pixels are provided among the plural pixels of the image pickup element 20. These methods include not only turning pixels on or off, but also changing sensitivity to light strength at different pixels to ensure the privacy of the human subject.

A first method comprises changing the area of the light receiving section 21 and the area of the aperture formed on the light shielding mask 23 for each pixel.

A second method comprises changing a reading property of the sensitivity information in the peripheral circuit 22 for each pixel.

A third method comprises pasting a filter having a different property or adding a substance having a different light transmission property for each pixel.

A fourth method comprises setting the computer-readable program of the image processor for signal processing so that output properties are different for different pixels.

Additional methods will now become apparent to those of ordinary skill in the art that may be also be used to alter the sensitivities of respective pixels.

In addition, according to the first embodiment, the functional pixels are arranged to form a plurality of groups wherein the pixels in each group are arranged to have an approximately circular shape. However, the pixels of each group may be arranged to have another shape, such as a rectangular or a polygonal shape. Then, this arrangement is not limited to those described above or shown in FIG. 4. However, the non-photographable light receiving sections 32 may be scattered and distributed on the photographable light receiving sections 31 such that the human subject cannot be recognized in detail from the image. In addition, the groups can be arranged at preselected positions, such as at periodic locations of a lattice.

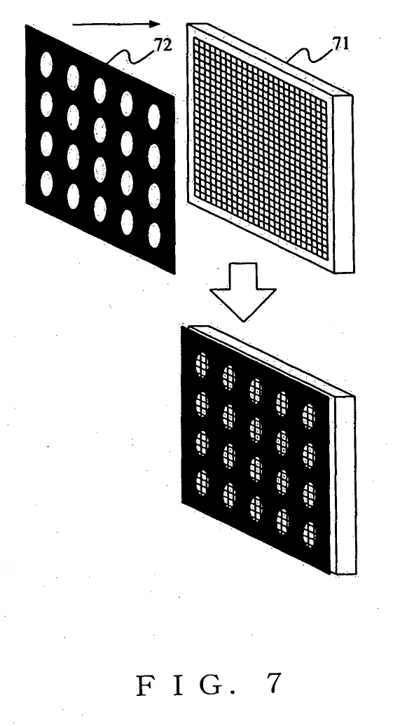

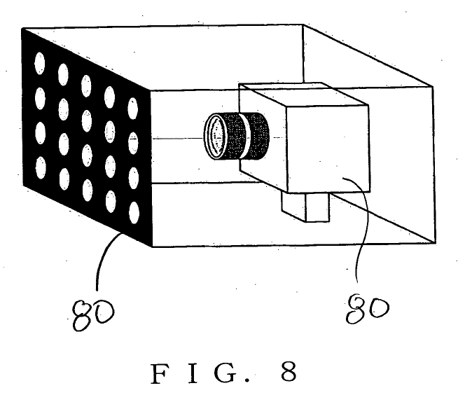

Next, a second embodiment is described with reference to FIGS. 7 and 8.

As shown in FIG. 3, in order to provide the functional light receiving sections 31 and the nonfunctional light receiving sections 32 for pixels and change the sensitivities thereof, a fine semiconductor processing technology may be required. However, it is not necessary to strictly control the function for each pixel and if an obstacle is disposed to provide photographable portions and non-photographable portions in the image pickup element 20 or the image pickup device 11, substantially the same function as that of the first embodiment can be accomplished.

FIG. 7 shows an exemplary embodiment of an obstacle such as a mask 72, provided with a plurality of circular apertures, that is fit on the light receiving surface of an image pickup element 71, such as a CCD element. Thus, if the mask 72 having the light transmitted through only specific apertures is coupled to the image pickup element 71, the image obtained by this image pickup element 71 is modified to ensure the privacy of the human, such as shown in FIG. 4 and FIG. 5.

In addition, a mask 80 having a photographable range set therein may be arranged outside of an image pickup device such as a CCD camera 82, as shown in FIG. 8.

Furthermore, a mask may be arranged inside or outside an optical system such as a lens of the image pickup device.

Furthermore, it is possible to arrange the mask at any position ranging from the light receiving surface of the image pickup element to the outside of the image pickup device.

In addition, the shape of the photographable range set by the apertures of the mask 72 or 80, or the like is not limited to a circle and various aperture shapes such as a rectangular and a polygon may be used. Furthermore, a plurality of masks can be arranged at preselected positions.

As described above, by providing the mask 72 or 80, or the like, for the image pickup element or device, it is possible to realize the image pickup device to ensure the privacy of the human subject without necessitating the fine processing technology.

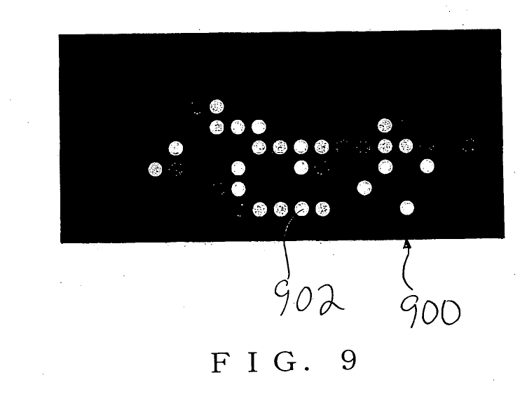

Next, a third embodiment will be described below with reference to FIG. 9.

The respiration measurement apparatus using the image pickup device may carry out the setting operation to adjust the direction and the position of the image pickup device while checking the image from the image pickup device with a display device such as a television (TV) monitor so that the image of the human subject is appropriately photographed. According to the image pickup device of the first and second embodiments, the schematic image of the human subject, as shown in FIG. 5, is obtained rather than a detailed image, such that, without using a comparatively large display device as the TV monitor, the display device described below can be used.

In the following description of an embodiment of display device 900 with reference to FIG. 9, it is assumed that the respiration measurement processing is carried out in groups of the functional pixels or each of the photographable image areas according to the first and second embodiment by any suitable conventional method.

In the display device 900 illustrated in FIG. 9, indicator lights 902, shown as white circles, such as light emitting diodes (LED), are arranged in a lattice and an individual pixel or one of the indicator lights 902 of the display device corresponds to an individual one of the functional pixel groups or the photographable image areas. If an illumination strength of each indicator light 902 is to be adjusted according to the strength (the brightness or the luminance) of the change of image detected in the functional pixel group or the photographable image area, it is possible to set the image pickup device that is appropriate for measurement of respiration by adjusting the direction and the position of the image pickup device so as to cause the group of indicator lights 902 to emit the bright light located at a center of the display device 900.

The display device 900 described above can be manufactured inexpensively and can be conveniently used. Furthermore, the respiration measurement apparatus comprising the display device 900 described above does not obtain the private visual information. Thus, this display device 900 is capable of appeasing the privacy concerns of the human subject.

In addition, in operation, the display device 900 is not limited to use in conjunction with the respiration measurement apparatus to ensure the privacy of the human subject by the image pickup device having the photographable image area and the non-photographable image area arranged as the first and second embodiments. For example, if the detection results of the change of image of the specific image area are unified by addition or the like and outputted, the display device 900 of the present embodiment can even be used in a respiration measurement apparatus using a conventional image pickup device.

Furthermore, the constitutional elements such as the indicator lights 902 of the display device 900 or other elements are not limited to the LED's, but may comprise other light emitting devices, such as one or more of an electric bulb, a liquid crystal element, a plasma display panel (PDP), and an organic electro luminescent (EL) element. In addition, the arrangement is not limited to the lattice, and the number and the arrangement thereof can be arbitrarily set.

Next, a fourth embodiment will be described below.

The number of pixels (the light receiving sections) of the CCD element has been increased to arrive at megapixel resolutions in recent years. However, photographing the human subject by the CCD element at such megapixel resolutions may yield an undesirably detailed image. Therefore, the image is modified, such as shown in FIG. 4 and FIG. 5, to provide privacy for the human subject by providing the nonfunctional light receiving section as in the first and second embodiments.

Therefore, if the human subject is photographed by using the CCD element having a small number of pixels, it is possible to generate a desirable image that allows measurement of the human subject's respiration while ensuring the human subject's privacy, as shown in FIGS. 4 and 5. However, if the number of pixels is too small, the obtained image may not be useable to measure the respiration of the human subject at a desirable detail.

As a result, it has been found that the measurement can be analyzed by the image for protecting the right to privacy as shown in FIGS. 4 and 5 and having a resolution of the image photographing the human subject from several tens to several thousands of pixels. For example, the CCD element may comprise from about 100 to about 5,000 pixels to generate a useful image while ensuring a desirable degree of privacy for the human subject.

In the first to fourth embodiments, the images are sensed in a visible range of the electromagnetic spectrum. However, in a fifth embodiment, the image is sensed in an infrared range of the electromagnetic spectrum, permitting the image to be sensed in a dark area.

The respiration measurement apparatus described above is capable of automatically measuring respiration of a human being, such as during sleeping, which can be used to support the care of elderly persons or children, or screening of a pulmonary disease.

What is claimed is:

1. A respiration measurement apparatus comprising:
image pickup means to photograph a human subject and provide information representative of an image of the human subject;
image inputting means to input the image information of the photographed human subject;
change detecting means to detect a change of the image due to respiration of the human subject from the inputted image information; and
respiration waveform generating means to obtain a respiration waveform from a time series of the detected change of image,
wherein a plurality of non-photographable image areas is distributed in photographable image areas in the image pickup means, and the change detecting means detects the change of the image from only at least one of the photographable image areas.

2. The respiration measurement apparatus according to claim 1,
wherein the image pickup means includes an image pickup device including an image pickup element; and
wherein the plurality of non-photographable image areas correspond to a plurality of non-photographable pixel groups distributed in groups of photographable pixels with respect to pixels of the image pickup element of the image pickup device.

3. The respiration measurement apparatus according to claim 1,
wherein the image pickup means includes an image pickup device including an image pickup element; and
wherein the non-photographable image areas correspond to a plurality of pixel groups having lower sensitivities than photographable image areas and are
distributed in groups of the photographable pixels corresponding to the photographable image areas with respect to pixels of the image pickup element of the image pickup device.

4. The respiration measurement apparatus according to claim 1,
wherein an obstacle having a plurality of apertures to limit the photographable image area distributed thereon is fit to the pixels of the image pickup element of the image pickup device.

5. The respiration measurement apparatus according to claim 1,
wherein the image pickup means includes an image pickup device including an image pickup element; and
wherein an obstacle having a plurality of apertures to limit the photographable image area distributed thereon is arranged in an optical system of the image pickup element of the image pickup device.

6. The respiration measurement apparatus according to claim 1,
wherein the image pickup means includes an image pickup device; and
wherein an obstacle having a plurality of apertures to limit the photographable image area distributed thereon is arranged outside the image pickup device.

7. A respiration measurement apparatus comprising:
an image pickup device including an image pickup element to photograph a human subject and provide information representative of an image of the human subject;
image inputting means to input the image information of the photographed human subject;
change detecting means to detect a change of the image due to respiration of the human subject from the inputted image information; and
respiration waveform generating means to obtain a respiration waveform from a time series of the detected change of image,
wherein a resolution of the image photographed by the image pickup element of the image pickup device is defined from several tens to several thousands of pixels.

8. A respiration measurement apparatus comprising:
an image pickup device including an image pickup element to photograph a human subject and provide information representative of an image of the human subject;
image inputting means to input the image information of the photographed human subject;
change detecting means to detect a change of the image due to respiration of the human subject from the inputted image information; and
respiration waveform generating means to obtain a respiration waveform from a time series of the detected change of image,
wherein the respiration measurement apparatus includes display means to only display brightness information for individual pixels or for groups of pixels of the image pickup element in the image photographed by the image pickup device.

9. A respiration measurement apparatus comprising:
an image pickup device including an image pickup element to photograph a human subject and provide information representative of an image of the human subject;
a respiration measurement device to input the image information of the photographed human subject;
a processor, included in the respiration measurement device, to detect a change of the image due to respiration of the human subject from the inputted image information and to generate a respiration waveform from a time series of the detected change of the image;
wherein a plurality of non-photographable image areas are distributed in photographable image areas in the image pickup element of the image pickup device, and the processor detects the change of the image only from at least one of the photographable image areas.

10. A respiration measurement apparatus comprising:
an image pickup device including an image pickup element to photograph a human subject and provide information representative of an image of the human subject;
a respiration measurement device to input the image information of the photographed human subject;
a processor, included in the respiration measurement device, to detect a change of the image due to respiration of the human subject from the inputted image information and to generate a respiration waveform from a time series of the detected change of the image;
wherein a resolution of the image photographed by the image pickup element of the image pickup device is defined from several tens to several thousands of pixels.

11. A respiration measurement apparatus comprising:
an image pickup device including an image pickup element to photograph a human subject and provide information representative of an image of the human subject;
a respiration measurement device to input the image information of the photographed human subject;
a processor, included in the respiration measurement device, to detect a change of the image due to respiration of the human subject from the inputted image information and to generate a respiration waveform from a time series of the detected change of the image;
wherein the respiration measurement apparatus includes display means to only display brightness information for individual pixels or for groups of pixels of the image pickup element in the image photographed by the image pickup device.

12. A display device for displaying brightness information for a plurality of pixels or groups of pixels of an image pickup element in an image photographed by an image pickup device of a respiration measurement apparatus, the display device comprising:
a plurality of indicator lights arranged in a lattice, wherein the individual indicator lights correspond to the individual pixels or groups of pixels of the image pickup element.
